(19) United States
(12) Patent Application Publication
(10) Pub. No.: US 2009/0012577 A1
(43) Pub. Date: Jan. 8, 2009

(54) APPARTUS AND METHOD FOR TREATING HEADACHE AND/OR FACIAL PAIN

(75) Inventors: Ali R. Rezai, Shaker Heights, OH (US); Mehdi M. Ansarinia, Las Vegas, NV (US); Scott B. Kokones, Cleveland, OH (US); Charles P. Steiner, Cleveland, OH (US); Keith R. Carlton, Cleveland, OH (US); Daniel N. Kelsch, Fairview Park, OH (US); Richard Drake, Shaker Heights, OH (US); Jennifer McBride, Cleveland, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/130,059

(22) Filed: May 30, 2008

Related U.S. Application Data
(60) Provisional application No. 60/932,233, filed on May 30, 2007.

(51) Int. Cl. A61N 1/34

(52) U.S. Cl. 607/46

(57) ABSTRACT

An apparatus for treating headache and/or facial pain includes an electrical lead and having a distal end portion, a proximal end portion, and a channel extending between the distal and proximal end portions. The distal end portion has at least one electrode disposed thereon and at least one foldable tine for anchoring the distal tip adjacent a SPG, and the proximal end portion is adapted for connection to an energy delivery source.

### RELATED APPLICATION

This application claims priority from U.S. Provisional Patent Application Ser. No. 60/932,233, filed May 30, 2007, the entirety of which is hereby incorporated by reference.

### TECHNICAL FIELD

The present invention relates generally to an apparatus and method for treating pain, and more particularly to an implantable apparatus and method for treating headache and/or facial pain.

### BACKGROUND OF THE INVENTION

The International Headache Society (IHS) classifies cluster headaches as a primary headache disorder. These headaches are considered a severe type of headache, even more intense than a migraine attack. They have been called the “suicide headache” because many people have taken their lives either during an attack or in anticipation of an attack.

The term “cluster headache” refers to headaches that have a characteristic grouping of attacks. The headaches typically last between 15 to 180 minutes, and may occur up to six times per day. These headaches tend to occur daily for two weeks to three months and then can go into remission for months or years, only to recur. Further, it is not uncommon for a cluster headache to awaken a person from sleep one to two hours after going to bed. These nocturnal attacks have been known to be more severe than the daytime attacks.

There are two types of cluster headaches: episodic and chronic. In the episodic form, a person has one or more cluster cycles per year and, on occasion, may be free of headaches for a number of years. People with chronic cluster headaches have more than 50 weeks of headaches per year every year until the headache pattern changes.

The pain associated with a cluster headache is extremely intense and usually localized around the eye, temple, forehead and cheek. Occasionally, the headaches spread to the ipsilateral occipitocervical junction. Sufferers are incapacitated during these headaches, which are usually sharp in nature. Typically, there are associated autonomic features including lacrimation, rhinorrhea, ptosis, conjunctival injection, periorbital edema, facial flushing and nasal congestion.

Fortunately, most cluster headaches are responsive to preventive and abortive medications. However, in refractory cases, sufferers may be on multiple medications including daily opioids and still have breakthrough pain. At times, these people feel desperate and despondent and may contemplate suicide.

The available treatments for cluster headache are limited. Because of the brief duration of an attack, the abortive treatment of these headaches is difficult. Often, the acute headache has disappeared before the patient arrives at the emergency department or physician's office to receive treatment. The most commonly used abortive agents are oxygen inhalation therapy and IMITREX injections. Other medications used in the prevention of cluster headaches include verapamil, lithium carbonate, divalproex sodium, corticosteroids, METHERGINE, melatonin, and topiramate.

In addition to these non-invasive medications (which can have severe side-effects), many invasive procedures have been performed with the intent of preventing cluster headaches. These procedures vary from least invasive (e.g., sphenopalatine blocks using lidocane or cocaine which provide 80%-85% relief of pain on a temporary basis) to much more invasive (e.g., surgical resection of the sphenopalatine ganglia, radiofrequency gangliorhizolysis, gamma knife radio surgery, and stimulation of the hypothalamus). These later procedures are very invasive, and most are non-reversible. The efficacy of these procedures is, at best, 50%-75% (less than 5 years). Finally, these procedures produce the chance for significant morbidity in terms of facial dysesthesia/anesthesia delarosa, corneal ulcers, and facial sensory loss.

### SUMMARY OF THE INVENTION

In one aspect of the present invention, an apparatus for treating headache and/or facial pain in a subject comprises an electrical lead having a distal end portion, a proximal end portion, and a channel extending between the distal and proximal end portions. The distal end portion is for positioning about a sphenopalatine ganglion (SPG), and includes a distal tip having at least one electrode disposed thereon. The distal end portion further includes at least one foldable tine for anchoring the distal tip adjacent the SPG, and the proximal end portion is adapted for connection to an energy delivery source.

In another aspect of the present invention, an apparatus for treating headache and/or facial pain in a subject comprises an electrical lead having a distal end portion, a proximal end portion, and a channel extending between the distal and proximal end portions. The distal end portion is for positioning about a SPG, and includes a distal tip having at least one electrode disposed thereon. The distal end portion further includes at least one foldable tine for anchoring the distal tip adjacent the SPG, and the proximal end portion is adapted for connection to an energy delivery source. The apparatus also includes a pre-formed steering stylet for guiding the distal end portion of the electrical lead around a posterior edge of a pterygomaxillary fissure. The steering stylet has proximal and distal end portions, and is insertable into the channel of the electrical lead. The apparatus additionally includes an introduction needle dimensioned to deliver the distal end portion of the electrical lead at a desired orientation within the pterygopalatine fossa. The introduction needle has a distal end portion, a proximal end portion, and a lumen extending between the distal and proximal end portions for receiving the electrical lead. Each of the distal and proximal end portions has a bent configuration to facilitate delivery of the distal tip of the electrical lead adjacent the SPG.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a perspective view of an apparatus comprising an introduction needle, a positioning lead, and a steering stylet for treating headache and/or facial pain constructed in accordance with the present invention;

FIG. 2 is an exploded cutaway view of the electrical lead in FIG. 1;

FIG. 3 a perspective view of a human skull showing the introduction needle of FIG. 1 being advanced toward the pterygopalatine fossa;

FIG. 4 is a cross-sectional view taken along Line 4-4 in FIG. 3 showing the introduction needle positioned adjacent the sphenopalatine ganglion (SPG);

FIG. 5 is a cross-sectional view taken along Line 4-4 in FIG. 3 showing the electrical lead being urged out of the introduction needle;

FIG. 6 is a cross-sectional view taken along Line 4-4 in FIG. 3 showing the electrical lead positioned adjacent the SPG;

FIG. 7 is a cross-sectional view taken along Line 4-4 in FIG. 3 showing electrical energy being delivered to the electrical lead via an implantable pulse generator (IPG); and

FIG. 8 is a perspective view of an upper human torso showing the apparatus of FIG. 1 fully implanted and connected to the IPG.

### DETAILED DESCRIPTION

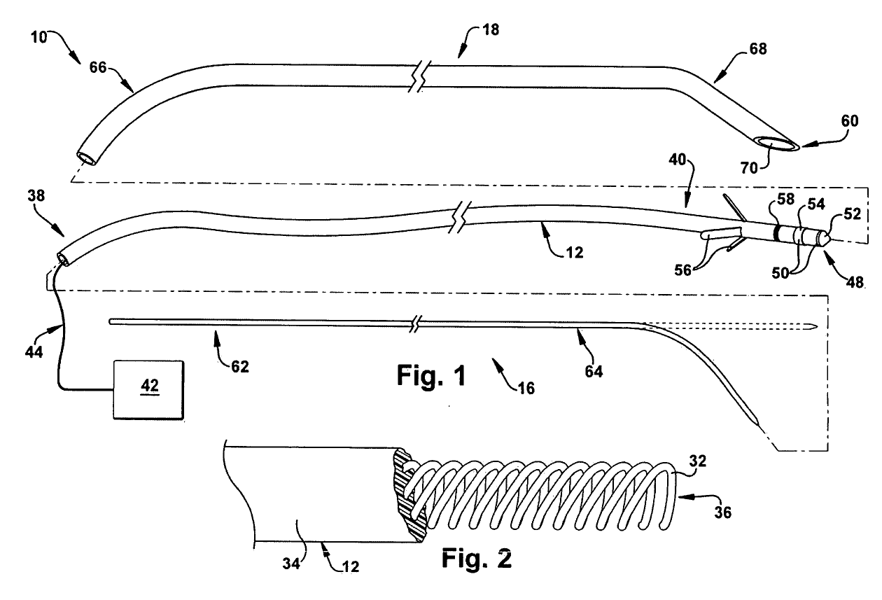

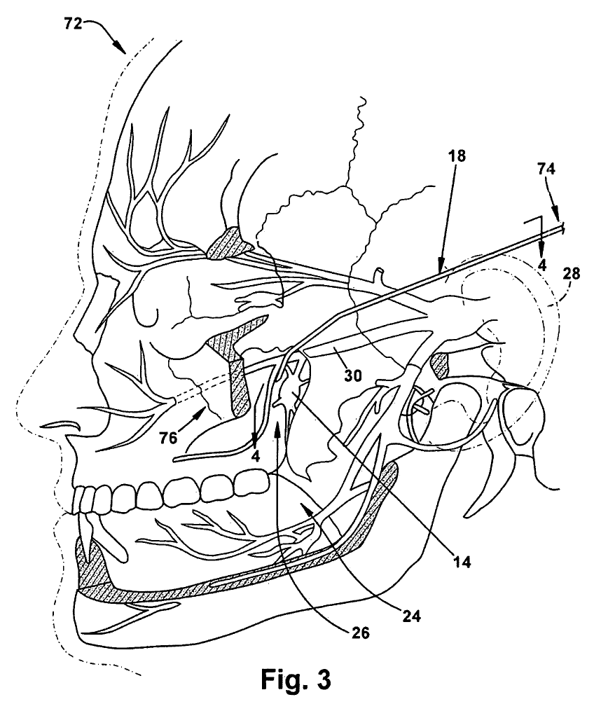

The present invention relates generally to an apparatus and method for treating pain, and more particularly to an implantable apparatus and method for treating headache and/or facial pain. As representative of the present invention, FIG. 1 illustrates an apparatus 10 for treating chronic or acute headache and/or facial pain. The apparatus 10 comprises an electrical lead 12 for positioning about a sphenopalatine ganglion 14 (SPG; also called the pterygopalatine ganglion) (FIG. 3), a pre-formed steering stylet 16 for guiding the electrical lead, and an introduction needle 18 for delivering the electrical lead to the SPG. As discussed in greater detail below, it will be appreciated that the present invention may be employed to treat a variety of other chronic or acute medical conditions besides headache and/or facial pain including, but not limited to, pain, movement disorders, epilepsy, cerebrovascular diseases, autoimmune diseases, sleep disorders, autonomic disorders, urinary bladder disorders, abnormal metabolic states, disorders of the muscular system, and neuropsychiatric disorders.

Unless otherwise defined, all technical terms used herein have the same meaning as commonly understood by one of ordinary skill in the art to which the present invention pertains.

In the context of the present invention, the term “headache” as used herein refers to migraines, tension headaches, cluster headaches, trigeminal neuralgia, secondary headaches, tension-type headaches, chronic and epsisodic headaches, medication overuse/rebound headaches, chronic paroxysmal hemicrinia headaches, hemicranias continua headaches, post-traumatic headaches, post-herpetic headaches, vascular headaches, reflex sympathetic dystrophy-related headaches, crvicalgia headaches, caroidynia headaches, sciatica headaches, trigeminal headaches, occipital headaches, maxillary headaches, diary headaches, paratrigeminal headaches, petrosal headaches, Sluder's headache, vidian headaches, low CSF pressure headaches, TMJ headaches, causalgia headaches, myofascial headaches, all primary headaches (e.g., primary stabbing headache, primary cough headache, primary exertional headache, primary headache associated with sexual activity, hypnic headache, and new daily persistent headache), all trigeminal autonomic cephalagias (e.g., episodic paroxysmal hemicranias, SUNCT, all probable TACs, and SUNA), chronic daily headaches, occipital neuralgia, atypical facial pain, neuropathic trigeminal pain, and miscellaneous-type headaches.

As used herein, the term “cluster headache” refers to extremely painful and debilitating headaches that occur in groups or clusters. Cluster headaches can include cluster-type headaches, histamine headaches, histamine cephalalgia, Raedar's syndrome, and sphenopalatine neuralgia.

As used herein, the term “migraine” refers to an intense and disabling episodic headache typically characterized by severe pain in one or both sides of the head. Migraines can include, but are not limited to, migraine without aura, migraine with aura, migraine with aura but without headache, menstrual migraines, variant migraines, transformed migraines, complicated migraines, hemiplegic migraines, atypical migraines, chronic migraines, basilar-type migraines, childhood periodic syndromes that are commonly precursors of migraine (e.g., abdominal, cyclic vomiting, BPV, etc.), status migrainous, and all types of probable migraines.

As used herein, the term “facial pain” refers to direct pain that typically involves nerves supplying the face or, alternatively, indirect (referred) pain from other structures in the head, e.g., blood vessels. The pain may be related to headache (e.g., migraine), muscular syndromes such as TMJ, and herpetic or rheumatic disease or injury.

As used herein, the terms “modulate” or “modulating” refer to causing a change in neuronal activity, chemistry and/or metabolism. The change can refer to an increase, decrease, or even a change in a pattern of neuronal activity. The term may refer to either excitatory or inhibitory stimulation, or a combination thereof, and may be at least electrical, biological, magnetic, optical or chemical, or a combination of two or more of these. The term “modulate” can also be used to refer to a masking, altering, overriding, or restoring of neuronal activity.

As used herein, the term “subject” refers to any warm-blooded organism including, but not limited to, human beings, pigs, rats, mice, dogs, goats, sheep, horses, monkeys, apes, rabbits, cattle, etc.

As used herein, the term “prevent” shall have its plain and ordinary meaning to one skilled in the art of pharmaceutical or medical sciences. For example, “prevent” can mean to stop or hinder a headache.

As used herein, the terms “treat” or “treating” shall have their plain and ordinary meaning to one skilled in the art of pharmaceutical or medical sciences. For example, “treat” and “treating” can mean to prevent or reduce a headache.

As used herein, the term “medical condition” refers to pain, movement disorders, epilepsy, cerebrovascular diseases, autoimmune diseases, sleep disorders, autonomic disorders, urinary bladder disorders, abnormal metabolic states, disorders of the muscular system, infectious and parasitic diseases (as provided in ICD-9 codes 1-139), neoplasms (as provided in ICD-9 codes 140-239), endocrine, nutritional and metabolic diseases, and immunity diseases (as provided in ICD-9 codes 240-279), diseases of the blood and blood-forming organs (as provided in ICD-9 codes 280-289), mental disorders (as provided in ICD-9 codes 290-319), diseases of the nervous system (as provided in ICD-9 codes 320-359), diseases of the sense organs (as provided in ICD-9 codes 360-389), diseases of the circulatory system (as provided in ICD-9 codes 390459), diseases of the respiratory system (as provided in ICD-9 codes 460-519), diseases of the digestive system (as provided in ICD-9 codes 520-579), diseases of the genitourinary system (as provided in ICD-9 codes 580-629), diseases of the skin and subcutaneous tissue (as provided in ICD-9 codes 680-709), diseases of the musculoskeletal system and connective tissue (as provided in ICD-9 codes 710-739), congenital anomalies (as provided in ICD-9 codes 740-759), certain conditions originating in the perinatal period (as provided in ICD-9 codes 760-779), and symptoms, signs, and ill-defined conditions (as provided in ICD-9 codes 780-799).

Pain treatable by the present invention can be caused by conditions including, but not limited to, migraine headaches, including migraine headaches with aura, migraine headaches without aura, menstrual migraines, migraine variants, atypical migraines, complicated migraines, hemiplegic migraines, transformed migraines, and chronic daily migraines, episodic tension headaches, chronic tension headaches, analgesic rebound headaches, episodic cluster headaches, chronic cluster headaches, cluster variants, chronic paroxysmal hemicranias, hemicrania continua, post-traumatic headache, post-traumatic neck pain, post-herpetic neuralgia involving the head or face, pain from spine fracture secondary to osteoporosis, arthritis pain in the spine, headache related to cerebrovascular disease and stroke, headache due to vascular disorder, reflex sympathetic dystrophy, cervicalgia (which may be due to various causes, including, but not limited to, muscular, discogenic, or degenerative, including arthritic, posturally related, or metastatic), glossodynia, carotidynia, cricoidynia, otalgia due to middle ear lesion, gastric pain, sciatica, maxillary neuralgia, laryngeal pain, myalgia of neck muscles, trigeminal neuralgia (sometimes also termed tic douloureux), post-lumbar puncture headache, low cerebro-spinal fluid pressure headache, temporomandibular joint disorder, atypical facial pain, ciliary neuralgia, paratrigeminal neuralgia (sometimes also termed Raeder's syndrome); petrosal neuralgia, Eagle's syndrome, idiopathic intracranial hypertension, orofacial pain, myofascial pain syndrome involving the head, neck, and shoulder, chronic migraneous neuralgia, cervical headache, paratrigeminal paralysis, SPG neuralgia (sometimes also termed lower-half headache, lower facial neuralgia syndrome, Sluder's neuralgia, and Sluder's syndrome), carotidynia, vidian neuralgia, causalgia, and/or a combination of the above.

Movement disorders treatable by the present invention may be caused by conditions including, but not limited to, Parkinson's disease, cerebropalsy, dystonia, essential tremor, and hemifacial spasms.

Epilepsy treatable by the present invention may be, for example, generalized or partial.

Cerebrovascular disease treatable by the present invention may be caused by conditions including, but not limited to, aneurysms, strokes, and cerebral hemorrhage.

Autoimmune diseases treatable by the present invention include, but are not limited to, multiple sclerosis.

Sleep disorders treatable by the present invention may be caused by conditions including, but not limited to, sleep apnea and parasomnias.

Autonomic disorders treatable by the present invention may be caused by conditions including, but not limited to, gastrointestinal disorders, including but not limited to gastrointestinal motility disorders, nausea, vomiting, diarrhea, chronic hiccups, gastroesphageal reflux disease, and hypersecretion of gastric acid, autonomic insufficiency; excessive epiphoresis, excessive rhinorrhea; and cardiovascular disorders including, but not limited, to cardiac dysrythmias and arrythmias, hypertension, and carotid sinus disease.

Urinary bladder disorders treatable by the present invention may be caused by conditions including, but not limited to, spastic or flaccid bladder.

Abnormal metabolic states treatable by the present invention may be caused by conditions including, but not limited to, hyperthyroidism or hypothyroidism.

Disorders of the muscular system treatable by the present invention include, but are not limited to, muscular dystrophy, and spasms of the upper respiratory tract and face.

Neuropsychiatric or mental disorders treatable by the present invention may be caused by conditions including, but not limited to, depression, schizophrenia, bipolar disorder, and obsessive-compulsive disorder.

The present invention acts to suppress or prevent headache, facial pain, and/or other medical conditions by disrupting sensory signals passing through the autonomic nervous system, including pain signals, as the signals traverse or are generated in the SPG 14. The abnormal regulation of pain pathways, which may be a feature of headache, facial pain, and/or the medical conditions described above, can cause excitation or a loss of inhibition of those pathways resulting in an increased perception of pain. Direct electrical modulation of the SPG 14 can block the transmission of pain signals and stimulate inhibitory feedback of the pain pathways passing through the SPG, and thereby reduce or eliminate pain experienced by a subject. Similarly, electrical modulation of the SPG 14 can block the transmission of signals other than pain which can provoke or aggravate other undesirable sensations or conditions, such as nausea, bladder disorders, sleep disorders, or abnormal metabolic states.

A brief discussion of the pertinent neurophysiology is provided to assist the reader with understanding the present invention. The autonomic nervous system, which innervates pain pathways within the human body, consists of two divisions: the sympathetic and the parasympathetic nervous systems. The sympathetic and parasympathetic nervous systems are antagonistic in their action, balancing the other system's effects within the body. The sympathetic nervous system usually initiates activity within the body, preparing the body for action, while the parasympathetic nervous system primarily counteracts the effects of the sympathetic nervous system.

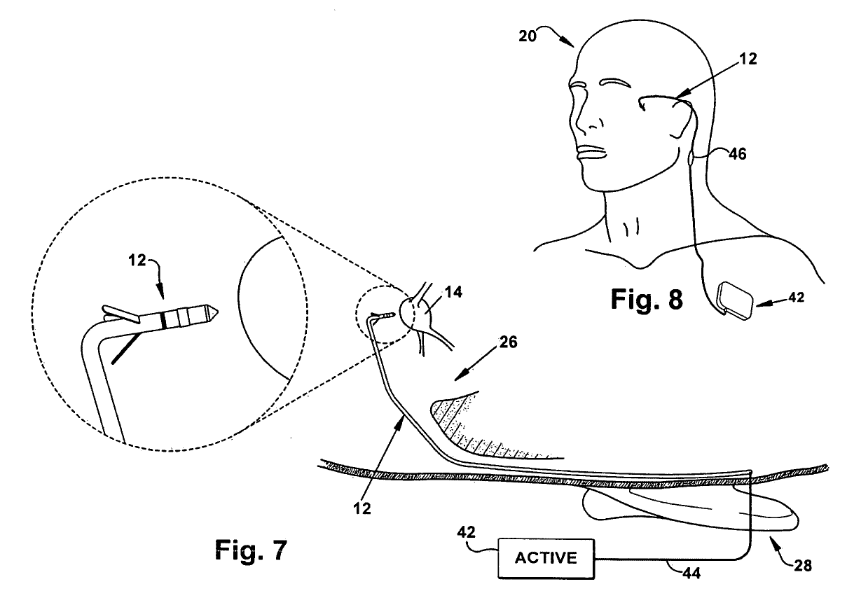

SPG 14 structures are located on both sides of a subject's head 20 (FIG. 8). The present invention may be applied to supply an electric current to the SPG 14 (FIG. 3) on either or both sides of a subject's head 20. With reference to FIGS. 3-8, it shall be assumed for the following discussion that the present invention is being applied to the left side of the subject's head 20.

Referring to FIG. 3, the SPG 14 is located behind the maxilla 24 in the pterygopalatine fossa 26 (PPF) posterior to the middle nasal turbinate (not shown in detail). The SPG 14 is surrounded by a layer of mucosal and connective tissue of less than five millimeters in thickness. The SPG 14 is part of the parasympathetic division of the autonomic nervous system. However, the SPG 14 has both sympathetic and parasympathetic nerve fibers, as well as sensory and visceral nerve fibers. The parasympathetic activity of the SPG 14 is mediated through the greater petrosal nerve (not shown), while the sympathetic activity of the SPG is mediated through the deep petrosal nerve (not shown), which is essentially an extension of the cervical sympathetic chain (not shown). Sensations generated by or transmitted through the SPG 14 include, but are not limited to, sensations to the upper teeth, feelings of foreign bodies in the throat, and persistent itching of the ear 28. Facial nerve and carotid plexuses (not shown) directly communicate sensory signals to the SPG 14, and cell bodies in the ventral horn of the thoracolumbar spinal cord (not shown) send fibers either directly or via cervical ganglion (not shown) to the SPG. The SPG 14 transmits sensory information, including pain, to the trigeminal system via the maxillary branch 30.

Referring to FIG. 1, an apparatus 10 for treating headaches and/or facial pain comprises an electrical lead 12 for positioning about the SPG 14 (FIG. 3), a pre-formed steering stylet 16 (FIG. 1) for guiding the electrical lead, and an introduction needle 18 for delivering the electrical lead to the SPG. The electrical lead 12 has an elongated, flexible configuration and is made of a biocompatible material, such as urethane. As shown in FIG. 2, the electrical lead 12 is comprised of two coradially coiled wires 32 surrounded by a biocompatible insulating jacket 34. The coradially coiled wires 32 are comprised of an electrically conductive material, such as platinum-iridium, and are at least partially coated with a fluoropolymer to facilitate fluoroscopic visualization of the electrical lead 12. Although the coiled wires 32 may be made of any electrically conductive material, it will be appreciated that platinum-iridium is preferred because of its excellent biocompatibility and high electrical impedance.

The length and diameter of the electrical lead 12 can be varied based on the particular clinical need(s) of the subject and/or the subject's neuroanatomy. For example, the electrical lead 12 may have a diameter of less than about 1 mm. The coradially coiled configuration of the wires 32 forms a channel 36 which extends between proximal and distal end portions 38 and 40 of the electrical lead 12. As described in more detail below, this configuration allows the steering stylet 16 to be received in the channel 36 and used to direct the electrical lead 12 when it first emerges from the introduction needle 18.

The proximal end portion 38 of the electrical lead 12 is adapted for connection to an energy delivery source 42. Although not shown in detail, the proximal end portion 38 includes a bipolar connector (not shown) for joining the electrical lead 12 to a connecting lead 44 which can be connected to the energy delivery source 42. The energy delivery source 42 can include any one or combination of internal, passive, or active energy delivery sources, such as radio frequency energy, X-ray energy, microwave energy, acoustic or ultrasound energy, such as focused ultrasound or high intensity focused ultrasound energy, light energy, electric field energy, thermal energy, magnetic field energy, and/or combinations of the same. Alternatively, the energy delivery source 42 can comprise a device capable of harvesting mechanical and/or thermodynamic energy from the body of a subject, such as a piezoelectric device. The energy delivery source 42 can be directly or indirectly (e.g., wirelessly) coupled to the electrical lead 12. In one example of the present invention, the energy delivery source 42 can include an implantable pulse generator capable of delivering electrical energy to the electrical lead 12. Examples of implantable pulse generators suitable for use in the present invention are well known in the art.

Lead failure and migration are significant concerns for deep brain stimulation (DBS) systems. Certain portions of electrical leads must endure significant mechanical fatigue loading. Repeated mechanical stress caused by the normal articulation of the neck, for example, can cause failure in OBS leads and extensions. Lead failure and migration can be eliminated by placing a lead-extension connector away from the soft tissue of the neck. Because the motion of the mandibular structure and muscles of mastication may cause failure and/or migration of the electrical lead 12 (especially immediately after implantation but before natural adhesions form to fix the electrical lead), a lead-extension connector 46 may be used to facilitate placement of the electrical lead and prevent unwanted migration of the electrical lead.

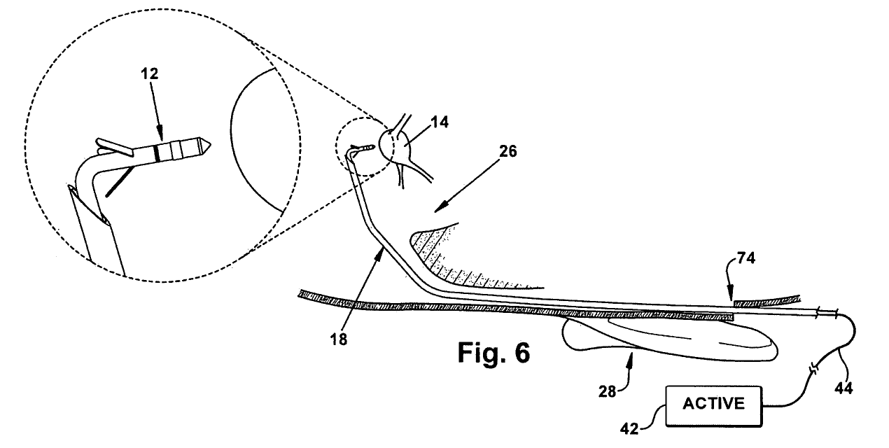

The distal end portion 40 of the electrical lead 12 is for positioning about the SPG 14. The distal end portion 40 is formed from a much stiffer urethane formulation than the rest of the electrical lead 12. The stiffer formulation causes the distal end portion 40 of the electrical lead 12 to obtain a bent configuration (FIG. 6) when urged from the introduction needle 18. As described in more detail below, the bent configuration allows the distal end portion 40 of the electrical lead 12 to hook around the PPF 26 so that a distal tip 48 of the electrical lead is positioned adjacent to or on the SPG 14.

As shown in FIG. 1, the distal end portion 40 of the electrical lead 12 includes a distal tip 48 having at least one electrode 50 disposed thereon. More particularly, the distal tip 48 includes first and second electrodes 52 and 54 comprised of 90/10 platinum-iridium, for example. Platinum-iridium is inert, exhibits high electrical impedance, and is extremely biocompatible. These properties make platinum-iridium an excellent selection for chronic implants. As illustrated in FIG. 1, the first electrode 52 has a conical shape and is disposed at the terminal end of the distal tip 48. The second electrode 54 has a band shape and is disposed proximal to the first electrode 52. It will be appreciated, however, that the electrodes 50 may have any shape and size, including, for example, a triangular shape, a rectangular shape, an ovoid shape, and are not limited to the shapes and sizes illustrated in FIG. 1. The distance between the first and second electrodes 52 and 54 may be varied as needed; however, a distance of about 3 mm is preferable.

To facilitate focal delivery of electrical energy to the SPG 14, the electrodes 50 may be arranged at the distal tip 48 of the electrical lead 12 to establish a desired electrode contact and coverage. Additionally or optionally, the entire surface area of the electrodes 50 may be conductive or, alternatively, only a portion of the surface area of the electrodes may be conductive. By modifying the conductivity of the surface of the electrodes 50, the surface area of the electrodes that contacts the SPG 14 may be selectively modified to facilitate focal delivery of electrical energy to the SPG.

It will be appreciated that electrode 50 configurations other than those illustrated in FIGS. 1-7 and described herein may also be used. For example, where the distal tip 48 of the electrical lead 12 has a flattened configuration (not shown), an electrode 50 could be located at the flattened distal tip so that electric current can be directed towards the SPG 14. Alternatively, the electrical lead 12 may have a bipolar configuration (e.g., the distal tip 48 of the electrical lead 12 may have a split tip design) that allows delivery of electric current to the SPG 14 using a guarded cathode (not shown). Moreover, it should be appreciated that the configuration of the electrode 50 can be identical or similar to electrodes used for DBS. DBS electrodes are known in the art and can include, for example, those disclosed in U.S. Patent Pub. No. 2008/0103547 A1 and U.S. Pat. Nos. 7,285,118 and 5,938,688,

The distal end portion 40 of the electrical lead 12 further comprises at least one foldable tine 56 for anchoring the distal tip 48 adjacent to, on, or inside of the SPG 14. As shown in FIG. 1, three tines 56 are operably secured to the distal end portion 40 of the electrical lead 12. It will be appreciated that any number of tines 56 may be operably secured to the electrical lead 12. The tines 56 are foldable so that each tine obtains a flattened configuration when the electrical lead 12 is disposed in the introduction needle 18. When the distal end portion 40 of the electrical lead 12 is extruded from the introduction needle 18, the tines 56 flare or spring radially outward to anchor in the surrounding tissue. Alternatively, the tines 56 may be operably connected to a deployment mechanism (not shown). The deployment mechanism may trigger the tines 56 to expand or unfold when the steering stylet 16 is removed from the channel 36 of the electrical lead 12. By anchoring the tines 56 in the surrounding tissue, the tines prevent migration of the electrical lead 12 until scar tissue can encapsulate and thereby secure the electrical lead.

The distal end portion 40 of the electrical lead 12 further includes at least one positioning band 58. The positioning band 58 is disposed proximal to the first and second electrodes 52 and 54 and is for indicating that the distal tip 48 of the electrical lead 12 has reached a distal tip 60 of the introduction needle 18. Additionally, the positioning band 58 includes at least one directional index (not shown) for indicating the position of the distal end portion 40 of the electrical lead 12 when the electrical lead is advanced through the introduction needle 18.

As shown in FIG. 1, the apparatus 10 further includes a pre-formed steering stylet 16 for guiding the distal end portion 40 of the electrical lead 12 around a posterior edge of a pterygomaxillary fissure (not shown in detail). The steering stylet 16 has an elongated, flexible configuration with proximal and distal end portions 62 and 64. The steering stylet 16 is made of a material having a high mechanical stiffness, such as stainless steel or tungsten. The steering stylet 16 is insertable into the channel 36 of the electrical lead 12 and, thus, has a diameter less than the diameter of the channel of the electrical lead. For example, the diameter of the steering stylet 16 may be about 0.15 mm.

The steering stylet 16 is preformed to facilitate positioning of the electrical lead 12. The steering stylet 16 is made with a preformed bend at the proximal end portion 62 to direct the electrical lead 12 out of the introduction needle 18 in a preferential direction, i.e., so that the steering stylet guides the electrical lead around the posterior edge of the pterygomaxillary fissure. To allow for insertion of the steering stylet 16 into the electrical lead 12, the steering stylet is sufficiently flexible to obtain a straightened configuration (indicated by dashed lines) as shown in FIG. 1. As discussed in more detail below, withdrawal of the steering stylet 16 from the electrical lead 12 allows the distal end portion 40 of the electrical lead to obtain the bent configuration (FIG. 6) and be positioned within the PPF 26.

As shown in FIG. 1, the apparatus 10 further includes an introduction needle 18 dimensioned to deliver the distal end portion 40 of the electrical lead 12 at a desired orientation within the PPF 26. The introduction needle 18 has a proximal end portion 66, a distal end portion 68, and a lumen 70 extending between the end portions for receiving the electrical lead 12. Each of the proximal and distal end portions 66 and 68 has a bent configuration that allows the introduction needle 18 to conform to the anatomy of the skull 72 and facilitate delivery of the distal tip 48 to the SPG 14. As shown in FIGS. 3-6, for example, the bent configuration of the proximal and distal end portions 66 and 68 allows the introduction needle 18 to pass along the temporal aspect of the skull 72 and curve medially toward the PPF 26.

The introduction needle 18 has a rigid configuration and can be made of a biocompatible, medical grade material, such as stainless steel. The distal end portion 68 of the introduction needle 18 includes a sharpened distal tip 60 for penetrating tissue. The proximal end portion 66 can include a handle (not shown) so that sufficient force and control can be used to position the introduction needle 18. The handle may include a channel aligned with the lumen 70 of the introduction needle 18 so that the electrical lead 12 can be easily inserted into the introduction needle during implantation.

Another embodiment of the present invention includes a method for treating headache and/or facial pain in a subject. To treat a subject suffering from refractory cluster headaches, for example, the neuroanatomy of the subject is first determined using known imaging techniques (e.g., MRI, CT, ultrasound, X-ray, fluoroscopy, or combinations thereof. In particular, the anatomy of the subject's skull, including the position of the SPG 14, is determined prior to implantation of an electrical lead 12.

To facilitate placement of the electrical lead 12, a scanning apparatus (not shown), such as a CT scan or fluoroscope is used to monitor the surgical procedure during localization of the SPG 14. For clarity, the present invention will be described here using a fluoroscope, but it should be understood that the present invention can be readily adapted for use with other imaging modalities, such as a CT scan. The subject is placed supine on a fluoroscopy table, with the subject's nose pointing vertically. The subject's head 20 is then fixed in place on the fluoroscopy table using, for example, a strip of adhesive tape. The fluoroscope (e.g., a fluoroscopy C-arm unit) is then adjusted to a straight lateral position.

After assessing the neuroanatomy of the subject and preparing the subject on the fluoroscopy table, the implant procedure begins with a small incision at a puncture point 74 over the ipsilateral temporal lobe, slightly superior to the ear 28. The incision goes down through the first two layers of the subject's scalp to the superficial temporal facia. Next, an obturator (not shown) is placed at the distal tip 60 of the introduction needle 18, and the introduction needle is inserted at the puncture point 74 and angled anteriomedially. The introduction needle 18 is advanced through the temporal fascia and temporalis muscle down to the periosteum of the temporal bone.

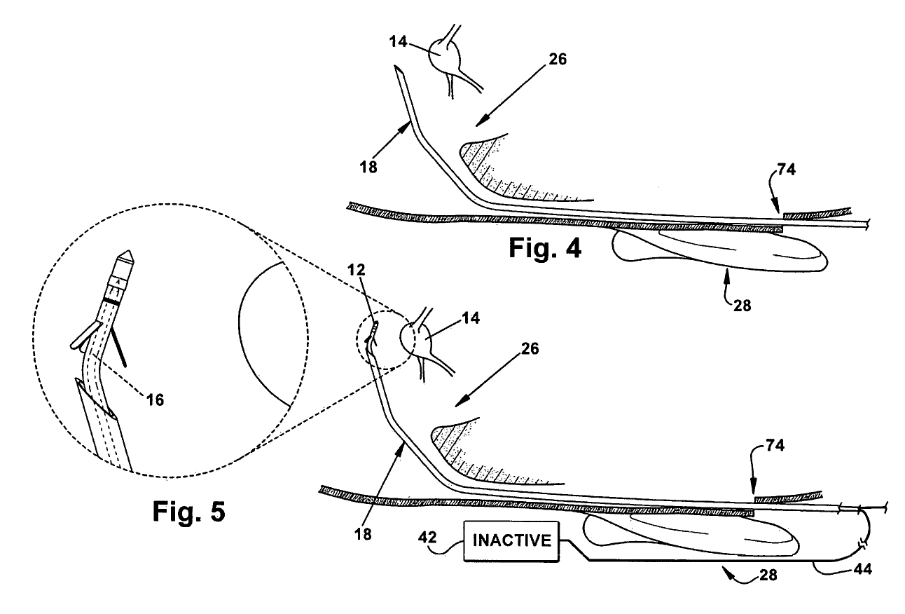

As shown in FIGS. 3-4, the introduction needle 18 is then advanced along the periosteum inferiorly towards the junction of the zygomatic arch and the temporal bone. The introduction needle 18 should ride along the top of the attachment point of the zygomatic arch 76 (shown in partial) to the temporal bone. Following this path, the introduction needle 18 is advanced towards the posterior surface of the maxilla 24. As the introduction needle 18 is advanced, the proximal end portion 66 of the introduction needle is curved towards the PPF 26 (FIG. 4). The introduction needle 18 should be stopped just posterior to the PPF 26.

Next, the obturator of the introduction needle 18 is removed. The electrical lead 12 is then inserted into the proximal end portion 66 of the introduction needle 18 and advanced so that the distal tip 48 of the electrical lead is at the distal tip 60 of the introduction needle. Positioning the distal tip 48 of the electrical lead 12 at the distal tip 60 of the introduction needle 18 is noted on fluoroscopy by the positioning band (i.e., the index marking should be positioned toward the skull 72). The steering stylet 16 is then inserted into the channel 36 of the electrical lead 12, and the distal end portion 64 of the steering stylet advanced to the distal end portion 40 of the electrical lead.

As shown in FIG. 5, the electrical lead 12 is then advanced so that the distal end portion 40 is extruded beyond the distal tip 66 of the introduction needle 18. At or about the same time that the electrical lead 12 is being extruded from the introduction needle, the steering stylet 16 is slowly withdrawn from the channel 36 of the electrical lead. This allows the distal tip 48 of the electrical lead 12 to curve toward the PPF 26 as the electrical lead is advanced. The electrodes 50 are then positioned so that one or both of the electrodes is in electrical contact with the SPG 14. By “electrical contact” it is meant that when electric current is delivered to the electrodes 50, deplorization of at least one nerve comprising the SPG 14 is elicited. For example, one or both of the electrodes 50 can be placed directly on a surface of the SPG 14, within all or just a portion of the SPG, or in close proximity to the SPG but without being in direct contact with the SPG.

When the distal tip 48 of the electrical lead 12 is positioned on or adjacent the SPG 14, the steering stylet 16 is entirely withdrawn from the electrical lead. Removal of the steering stylet 16 actuates the deployment mechanism and deploys the foldable tines 56 so that the distal tip 48 of the electrical lead 12 is securely positioned in the anterior portion of the temporalis muscle (not shown) (FIG. 6). It should be appreciated that electrical activity in the SPG 14 can be additionally or optionally modulated by mechanically and/or chemically disrupting the SPG. For example, placement of a portion of the distal tip 48 onto or into the SPG 14 may be sufficient to mechanically modulate (e.g., disrupt) SPG function without delivery of electric current to the electrodes 50. Additionally, the electrical lead 12 may be configured to selectively deliver a chemical or biological agent (e.g., Botulinum toxin) to chemically modulate SPG 14 function.

If it has not already been done, the proximal end portion 38 of the electrical lead 12 is then connected to an energy delivery source 42 so that electrical energy can be delivered to the electrodes 50. The electrodes 50 and/or the energy delivery source 42 are/is controllable to produce output signals which can be varied in voltage, frequency, pulse width, current, and intensity. Further, the energy delivery source 42 may also be controllable so that the controller can produce both positive and negative current flow from the electrodes 50, stop current flow from the electrodes, or change the direction of current flow from the electrodes. The electrodes 50 can also have the capacity for variable output, linear output, and short pulse width. The electrodes 50 should be anchored securely at the site of implantation so that the output signals produced by the electrodes will consistently modulate the same region(s) of the SPG 14.

As the exact parameters of effective SPG 14 neuromodulation may vary between subjects, the electrodes 50 and/or energy delivery source 42 are/is controllable so that the electrical signal can be remotely adjusted to desired settings and retrieval of the electrical lead 12 from the subject is not necessary to adjust the subject's therapy. Remote control of the output signal can be affected, for example, using either conventional telemetry with an implanted pulse generator or, alternatively, using an implanted radiofrequency receiver (not shown) coupled to an external transmitter (not shown). It should be understood that as related technologies advance, other modalities for remote control of the electrical lead 12 may be employed to adjust and modulate the parameters of electric current delivery.

When electrical energy is to be applied to the SPG 14, the electrodes 50 are controlled to produce an electronic current wave. For example, the current wave may comprise relatively high frequency pulses with low frequency amplitude modulation. While the exact parameters for electrical modulation are not yet known, and are likely to vary by subject based upon data known for stimulations performed on the brain, spinal cord, and cranial nerves, optimal settings for modulation of the SPG 14 may fall in the range of an intensity of about 0.1-20 V, a frequency of about 1-1000 Hz, and a pulse-width of about 25-1000 μs. Additionally, it may be effective to produce high frequency bursts of current on top of an underlying low frequency continuous stimulus.

It will be appreciated that electric current can be delivered to the electrodes 50 continuously, periodically, episodically, or a combination thereof. For example, electric current can be delivered in a unipolar, bipolar, and/or multipolar sequence or, alternatively, via a sequential wave, charge-balanced biphasic square wave, sine wave, or any combination thereof. Electric current can be delivered to the electrodes 50 all at once or, alternatively, to only one of the electrodes using a controller (not shown) and/or known complex practice, such as current steering.

When electrical energy is delivered to the electrical lead 12, the output signal of the electrodes 50 is directly applied to the SPG 14 and acts to suppress the pain experienced by the subject by “blocking” the SPG. As used herein, the terms “block”, “blocking”, and “blockade” refer to the disruption, modulation, and/or inhibition of nerve impulse transmissions. As unregulated and increased nerve transmission is essential for the body to propagate and recognize pain, blocking nerve impulse transmissions through the SPG 14 can diminish the pain experienced by the subject.

Upon delivery of electrical energy to the electrodes 50, the subject is asked to report any pain sensation. The position of the electrical lead 12, or frequency of electrical energy being delivered to the electrodes 50, may then be adjusted until the subject reports that he or she is substantially pain free. After determining the optimal location and electrical energy delivery parameters, the introducing needle 18 is withdrawn from the subject. If it has not been done so already, the energy delivery source 42 is then implanted in the subject so that the electrical lead 12 and the energy delivery source are positioned in the subject as shown in FIG. 8. It will be appreciated, however, that only a portion of the energy delivery source 42 and/or electrical lead 12 may be implanted within the subject. Methods for implanting energy delivery sources 42, such as implantable pulse generators, are known in the art.

After successful implantation of the electrical lead 12, the subject can alter the electrical stimulus at the earliest onset of a cluster headache. Thus, the present invention provides a fully implantable, minimally invasive subject-activated stimulation system for triggering limited duration neuromodulation treatment of the SPG 14 at the earliest onset of headache and/or facial pain.

It should be appreciated that the electrical lead 12 can be part of an open- or closed-loop system. In an open-loop system, for example, a physician or subject may, at any time, manually or by the use of pumps, motorized elements, etc. tailor treatment parameters such as pulse amplitude, pulse-width, pulse frequency, or duty cycle. Alternatively, in a closed-loop system, electrical parameters may be automatically tailored in response to a sensed symptom or a related symptom indicative of a headache or medical condition. In a closed-loop feedback system, at least one sensor (not shown) that senses a symptom of the body can be a part of the electrical lead 12 or, alternatively, remotely placed at a bodily location.

From the above description of the invention, those skilled in the art will perceive improvements, changes and modifications. Such improvements, changes, and modifications are within the skill of the art and are intended to be covered by the appended claims.

Having described the invention, we claim:

1. An apparatus for treating headache and/or facial pain in a subject, said apparatus comprising:
an electrical lead having a distal end portion, a proximal end portion, and a channel extending between said distal and proximal end portions, said distal end portion for positioning about a sphenopalatine ganglion (SPG) and including a distal tip having at least one electrode disposed thereon, said distal end portion further including at least one foldable tine for anchoring said distal tip adjacent the SPG and said proximal end portion being adapted for connection to an energy delivery source.

2. The apparatus of claim 1, wherein said electrical lead is comprised of two coradially coiled wires surrounded by a biocompatible insulating jacket.

3. The apparatus of claim 2, wherein said two coradially coiled wires form said lumen that extends between said distal and proximal end portions of said electrical lead.

4. The apparatus of claim 2, wherein each of said two coradially coiled wires is made from an electrically conductive material and at least partially coated with a fluoropolymer.

5. The apparatus of claim 1, wherein said distal end portion of said electrical lead is movable from a first straightened configuration to a second bent configuration.

6. The apparatus of claim 1, wherein said distal tip includes a first electrode and said distal end portion includes a second electrode spaced apart from and disposed proximal to said first electrode.

7. The apparatus of claim 1 further comprising:
a pre-formed steering stylet for guiding said distal end portion of said electrical lead around a posterior edge of a pterygomaxillary fissure, said steering stylet having proximal and distal end portions and being insertable into said channel of said electrical lead; and
an introduction needle dimensioned to deliver said distal end portion of said electrical lead at a desired orientation within the pterygopalatine fossa, said introduction needle having a distal end portion, a proximal end portion, and a lumen extending between said distal and proximal end portions for receiving said electrical lead, each of said distal and proximal end portions having a bent configuration to facilitate delivery of said distal tip of said electrical lead adjacent the SPG.

8. The apparatus of claim 7, wherein said distal end portion of said introduction needle includes a sharpened distal tip for penetrating tissue.

9. The apparatus of claim 1, wherein said distal end portion of said electrical lead further includes a positioning band disposed proximal to said at least one electrode, said positioning band for indicating that said distal tip of said electrical lead has reached said distal tip of said introduction needle.

10. The apparatus of claim 9, wherein said positioning band includes at least one directional index for indicating the position of said distal end portion of said electrical lead when said electrical lead is advanced through said introduction needle.

11. The apparatus of claim 7, wherein said distal end portion of said steering stylet has a first straightened configuration to facilitate insertion of said steering stylet into said channel of said electrical lead and a second bent configuration for guiding said distal end portion of said electrical lead around the posterior edge of the pterygomaxillary fissure

12. An apparatus for treating headache and/or facial pain in a subject, said apparatus comprising:
an electrical lead having a distal end portion, a proximal end portion, and a channel extending between said distal and proximal end portions, said distal end portion for positioning about a sphenopalatine ganglion (SPG) and including a distal tip having at least one electrode disposed thereon, said distal end portion further including at least one foldable tine for anchoring said distal tip adjacent the SPG and said proximal end portion being adapted for connection to an energy delivery source;
a pre-formed steering stylet for guiding said distal end portion of said electrical lead around a posterior edge of a pterygomaxillary fissure, said steering stylet having proximal and distal end portions and being insertable into said channel of said electrical lead; and
an introduction needle dimensioned to deliver said distal end portion of said electrical lead at a desired orientation within the pterygopalatine fossa, said introduction needle having a distal end portion, a proximal end portion, and a lumen extending between said distal and proximal end portions for receiving said electrical lead, each of said distal and proximal end portions having a bent configuration to facilitate delivery of said distal tip of said electrical lead adjacent the SPG.

13. The apparatus of claim 12, wherein said electrical lead is comprised of two coradially coiled wires surrounded by a biocompatible insulating jacket.

14. The apparatus of claim 13, wherein said two coradially coiled wires form said lumen that extends between said distal and proximal end portions of said electrical lead.

15. The apparatus of claim 13, wherein each of said two coradially coiled wires is made from an electrically conductive material and at least partially coated with a fluoropolymer.

16. The apparatus of claim 12, wherein said distal end portion of said electrical lead is movable from a first straightened configuration to a second bent configuration.

17. The apparatus of claim 12, wherein said distal tip includes a first electrode and said distal end portion includes a second electrode spaced apart from and disposed proximal to said first electrode.

18. The apparatus of claim 12, wherein said distal end portion of said introduction needle includes a sharpened distal tip for penetrating tissue.

19. The apparatus of claim 12, wherein said distal end portion of said electrical lead further includes a positioning band disposed proximal to said at least one electrode, said positioning band for indicating that said distal tip of said electrical lead has reached said distal tip of said introduction needle.

20. The apparatus of claim 19, wherein said positioning band includes at least one directional index for indicating the position of said distal end portion of said electrical lead when said electrical lead is advanced through said introduction needle.

21. The apparatus of claim 12, wherein said distal end portion of said steering stylet has a first straightened configuration to facilitate insertion of said steering stylet into said channel of said electrical lead and a second bent configuration for guiding said distal end portion of said electrical lead around the posterior edge of the pterygomaxillary fissure.
